(19) United States
(12) Patent Application Publication
(10) Pub. No.: US 2020/0367984 A1
(43) Pub. Date: Nov. 26, 2020

(54) ROBOTIC SURGICAL SYSTEMS INCLUDING TORQUE SENSORS

(71) Applicant: Covidien LP, Mansfield, MA (US)

(72) Inventors: William Peine, Ashland, MA (US); Andreas Tobergte, Muenchen (DE)

(21) Appl. No.: 16/959,727

(22) PCT Filed: Jan. 2, 2019
(86) PCT No.: PCT/US2019/012015

§ 371 Date: Jul. 2, 2020

Related U.S. Application Data
(60) Provisional application No. 62/613,543, filed on Jan. 4, 2018.

(51) Int. Cl. A61B 34/37; B25J 13/06; B25J 9/16; B25J 13/08; B25J 13/02

(52) U.S. Cl. CPC A61B 34/37; B25J 13/065; B25J 9/1689; B25J 13/085; A61B 34/35; B25J 13/025; B25J 9/1682; B25J 9/1666; B25J 9/1633

(57) ABSTRACT

Methods are provided for handling collisions of a robotic surgical system. Collision handling may include receiving a first handle input and a second input at a controller. Upon receiving the first and second handle input, a desired position of a robotic arm is calculated. A first output signal to move the robotic arm toward the desired position is transmitted in response to calculating the desired position. As the robotic arm moves toward the desired position, a force measurement is received. If the force measurement is greater than a predetermined threshold, the desired position is recalculated.

### BACKGROUND

Robotic surgical systems have been used in minimally invasive medical procedures. Some robotic surgical systems include a console supporting a robotic arm and at least one end effector (e.g. forceps or a grasping tool) mounted to the robotic arm. The robotic arm provides mechanical power to the surgical instrument for operation of the surgical instrument. In addition, the robotic arm may provide electrical communication with the surgical instrument for operation. Each robotic arm may include an instrument drive unit that is operatively connected to the surgical instrument and that contains at least one drive mechanism.

Robotic surgical systems often include a surgeon's console having a handle assembly for actuating the functions of a surgical instrument. These handle assemblies implement actuation either via direct mechanical translation of force exerted by a user or alternatively translate mechanical user force or actuation into control signals which, in turn, are actuated by one or more electromechanical components within the handle assembly.

Depending on the function data transmitted to the surgical instrument from the surgeon's console, the surgical instrument may make a minor adjustment of a few millimeters, or alternatively may move a significant distance in the surgical field. Repositioning functions and translation of the instruments may, from time to time, cause the surgical robot to collide with another surgical instrument, a surgical cavity opening, or with anatomical parts located in the surgical cavity.

Accordingly, it is desirable for methods and systems to be disclosed which improve upon methods of detecting and handling collisions between surgical robots and objects foreign to the surgical robot.

### SUMMARY

In accordance with an aspect of the present disclosure, a method of collision handling for a robotic surgical system in a controller of the robotic surgical system includes receiving a first handle input and a second handle input from a console of the robotic surgical system, calculating a desired position of a robotic arm of a surgical robot in response to receiving the first and second handle inputs, transmitting a first output signal to the surgical robot to move the robotic arm toward the desired position, receiving a force measurement from the surgical robot as the robotic arm moves towards the desired position, and recalculating the desired position of the robotic arm of the surgical robot when the force measurement is greater than a predetermined threshold.

In aspects, the method includes taking force measurements at a joint of the robotic arm.

In some aspects, the force measurement taken at a joint of the robotic arm is a torque measurement.

In aspects, the method further includes transmitting a second output signal to continue moving the robotic arm toward the desired position when the force measurement is less than the predetermined threshold.

In particular aspects, the method further includes receiving a subsequent force measurement and recalculating the desired position of the robotic arm in response to receiving the subsequent force measurement.

In certain aspects, recalculating the desired position further includes setting the desired position as a current position of the surgical robot when the subsequent force measurement is greater than the predetermined threshold.

According to aspects, the method may further include transmitting control signals to transmit force feedback to an input handle when the force measurement is greater than the predetermined threshold.

In aspects, transmitting control signals includes transmitting control signals to transmit at least one of haptic feedback, tactile feedback, or sensory feedback to the input handle when the force measurement is greater than the predetermined threshold.

According to another aspect of the present disclosure, a method of collision handling for a robotic surgical system in a controller of the robotic surgical system includes determining a desired position of a robotic arm, transmitting an output signal to move the robotic arm towards the desired position, receiving a force measurement from the robotic arm as the robotic arm moves towards the desired position, and scaling down the output signal to move a location of the desired position when the force measurement is greater than a predetermined threshold.

In aspects, the method may further include receiving a first input signal and a second input signal, and determining the desired position of the robotic arm in response to receiving the first and second input signals.

In some aspects, the method may further include transmitting additional output signals to move the robotic arm toward the desired position when the torque measurement is less than the predetermined threshold.

In particular aspects, the method may include transmitting control signals to apply force feedback to an input handle when the torque measurement is greater than the predetermined threshold.

According to yet another aspect of the present disclosure, a method of collision handling for a robotic surgical system with a controller of the robotic surgical system may include determining a desired position of a robotic arm, transmitting a first output signal to move the robotic arm towards the desired position, receiving a force measurement from the robotic arm as the robotic arm moves towards the desired position, and transmitting an altered output signal when the force measurement is greater than a predetermined threshold.

In aspects, the method may further include receiving a first handle input and a second handle input.

In some aspects, determination of the desired position of the robotic arm may further include determining the desired position of the robotic arm in response to receiving the first and second handle input.

In particular aspects, the method may include transmitting an altered output signal to cause the robotic arm to move to an altered desired position in response to receiving the altered output signal.

According to aspects, the method may include transmitting control signals to move the robotic arm towards the desired position when the torque measurement is less than the predetermined threshold.

In certain aspects, the method may further include transmitting control signals to apply force feedback to an input handle when the torque measurement is greater than the predetermined threshold.

Although embodiments of the present disclosure are described in detail with reference to the accompanying drawings for the purpose of illustration and description, it is to be understood that the disclosed embodiments are not to be construed as limited thereby. It will be apparent to those of ordinary skill in the art that various modifications and/or combinations to the foregoing embodiments may be made without departing from the scope of the present disclosure.

### BRIEF DESCRIPTION OF THE DRAWINGS

FIG. 1 is an illustration of a surgical system, in accordance with an embodiment of the present disclosure;

FIG. 2 is an illustration of a robotic cart or tower of the surgical system of FIG. 1;

FIG. 3 is a functional block diagram of the system architecture for controlling the surgical system of FIG. 1;

FIG. 4 is a flow diagram of a prior art torque control process;

FIG. 5 is a flow diagram of a controller-based torque control process in accordance with the present disclosure;

FIG. 6 is a flow diagram of a controller-based repositioning process in accordance with the present disclosure;

FIG. 7A is a position diagram of a robotic arm advancing toward a second position without detecting a collision;

FIG. 7B is a position diagram of a robotic arm advancing toward a desired position and repositioning the desired position in accordance with the controller-based repositioning process of FIG. 5 after detecting a collision;

FIG. 7C is a position diagram of a robotic arm advancing toward a desired position and adjusting a scaling factor in accordance with the controller-based repositioning process of FIG. 6 after detecting a collision;

FIG. 8 is a flow diagram of a controller-based process for tracking an input handle during a collision in accordance with the present disclosure; and

FIG. 9 is a position diagram of an input handle and a robotic arm translated over time after in accordance with the controller-based process of FIG. 8.

### DETAILED DESCRIPTION

Embodiments of the present disclosure are described in detail with reference to the drawings, in which like reference numerals designate identical or corresponding elements in each of the several views.

As used herein, the term “distal” refers to the portion of the component being described which is further from a clinician, and the term “proximal” refers to the portion of the component being described which is closer to the clinician.

The term “clinician” as used herein refers to a doctor, nurse, healthcare provider which may include support personnel, or other operators of the surgical system described herein.

The term “surgical field” as used herein refers to the space in which the surgical robot operates. Such space may include, but is not limited to, an operating room, surgical robot storage and maintenance facility, and other space in which the surgical robot is disposed for mechanical operation.

The term “collision” as used herein refers to the contact of an element of a robotic surgical system with an object in the surgical field. Such collisions may include, for illustrative purposes, collisions with a robotic surgical instrument and a patient, either within a surgical cavity, with a robotic surgical instrument opening located on the patient, or with exterior tissue. Collisions may further include collisions with elements of other robotic surgical systems, other physical devices, and objects located in the surgical field.

The present disclosure relates to the alteration ofjoint position commands sent from a controller after detecting increases in torque beyond predetermined thresholds during a robotic surgical procedure. Additionally, the present disclosure relates to the scaling of motion as a controller receives torque measurements indicative of a collision between the robotic surgical system and a foreign object.

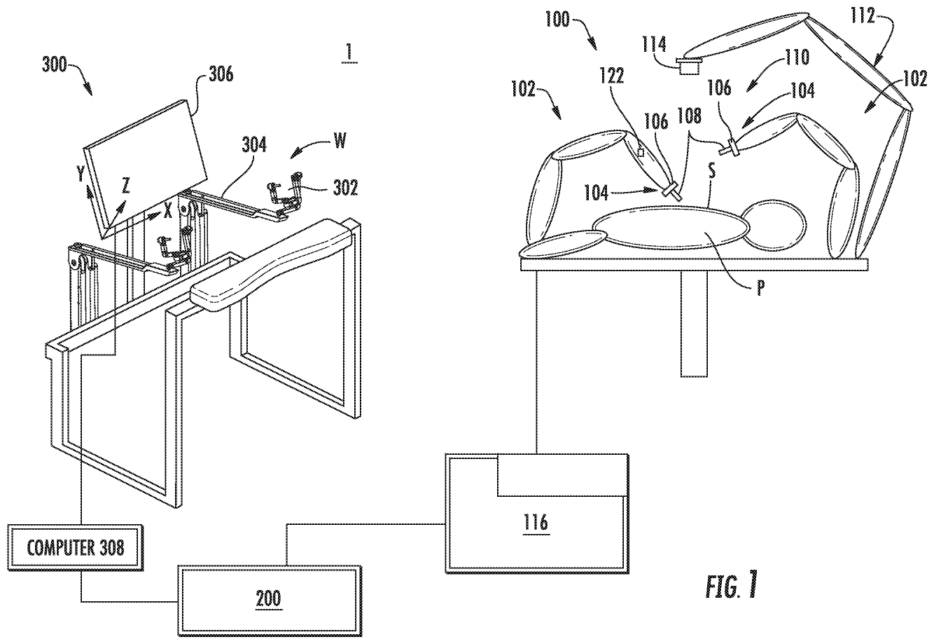

Referring to FIG. 1, a robotic surgical system 1 in accordance with the present disclosure is shown generally as a surgical robot 100, a controller 200, and a user interface or console 300 including a display 306. The surgical robot 100 generally includes a robotic cart or tower 116 which further includes linkages 112. The linkages 112 moveably support an end effector or tool 108 configured to act on tissue. The robotic arms 102 may be in the form of linkages 112, each robotic arm 102 having an end 104 that supports the end effector or tool 108. In addition, the ends 104 of the robotic arms 102 may include an imaging device 106 to image a surgical site “S”, as well as motor mechanisms 122 to apply force to joints “J” of the robotic arm and/or to actuate the tools 108.

The console 300 is in communication with tower 116 via the controller 200. The console 300 includes a display 306 which is configured to display three-dimensional images which may include data captured by imaging devices 106, 114 that are positioned about the surgical theater (e.g., an imaging device positioned within the surgical site “S”, an imaging device positioned adjacent the patient “P”, and/or an imaging device 114 supported by a distal portion of a robotic arm 102). The imaging devices (e.g., imaging devices 106, 114) may capture visual images, infra-red images, ultrasound images, X-ray images, thermal images, and/or any other known real-time images of the surgical site “S”. The imaging devices 106, 115 transmit captured imaging data to the processing unit 206 which creates three-dimensional images of the surgical site “S” in real-time from the imaging data and transmits the three-dimensional images to the display 306 for display.

The console 300 also includes input handles 302 which are supported on control arms 304 which allow a clinician to manipulate the surgical robot 100 (e.g., move the robotic arms 102, the ends 104 of the robotic arms 102, and/or the tools 108). Each of the input handles 302 is in communication with the processing unit 206 to transmit control signals thereto and to receive feedback signals therefrom. Additionally or alternatively, each of the input handles 302 may allow the surgeon to manipulate (e.g., clamp, grasp, fire, open, close, rotate, thrust, slice, etc.) the tools 108 supported at the ends 104 of the robotic arms 102.

With continued reference to FIG. 1, each of the input handles 302 is moveable through a predefined workspace “W” to move the ends 104 of the robotic arms 102, e.g., tools 108, within a surgical site “S”. The three-dimensional images on the display 306 are orientated such that the movement of the input handles 302 moves the ends 104 of the robotic arms 102 as viewed on the display 306. The three-dimensional images remain stationary while movement of the input handles 302 is scaled to movement of the ends 104 of the robotic arms 102 within the three-dimensional images. To maintain an orientation of the three-dimensional images, kinematic mapping of the input handles 302 is based on a camera orientation relative to an orientation of the ends 104 of the robotic arms 102. The orientation of the three-dimensional images on the display 306 may be mirrored or rotated relative to view from above the patient “P”. In addition, the size of the three-dimensional images on the display 306 may be scaled to be larger or smaller than the actual structures of the surgical site “S” permitting a clinician to have a better view of structures therein. As the input handles 302 are moved, the tools 108 are moved within the surgical site “S” as detailed below. Movement of the tools 108 may also include movement of the ends 104 of the robotic arms 102 which support the tools 108.

For a detailed discussion of the construction and operation of a robotic surgical system 1, reference may be made to U.S. Pat. No. 8,828,023, the entire contents of which are incorporated herein by reference.

The movement of the tools 108 is scaled relative to the movement of the input handles 302. When the input handles 302 are moved within the predefined workspace “W”, the input handles 302 send control signals to the processing unit 206. The processing unit 206 analyzes the control signals to move the tools 108 in response to the control signals. The processing unit 206 transmits scaled control signals to the tower 116 to move the tools 108 in response to the movement of the input handles 302. The processing unit 206 scales the control signals by dividing an Input_{distance} (e.g., the distance moved by one of the input handles 302) by a scaling factor S_{F} to arrive at a scaled Output_{distance} (e.g., the distance that one of the ends 104 is moved). The scaling factor S_{F} is in a range between about 1 and about 10 (e.g., 3). This scaling is represented by the following equation:

Output_{distance}=Input_{distance}/S_{F}

It will be appreciated that the larger the scaling factor S_{F} the smaller the movement of the tools 108 relative to the movement of the input handles 302.

For a detailed description of scaling movement of the input handle 302 along the X, Y, and Z coordinate axes to movement of the tool 108, reference may be made to commonly owned International Patent Application Serial No. PCT/US2015/051130, filed Sep. 21, 2015, and International Patent Application No. PCT/US2016/14031, filed Jan. 20, 2016, the entire contents of each of these disclosures is herein incorporated by reference.

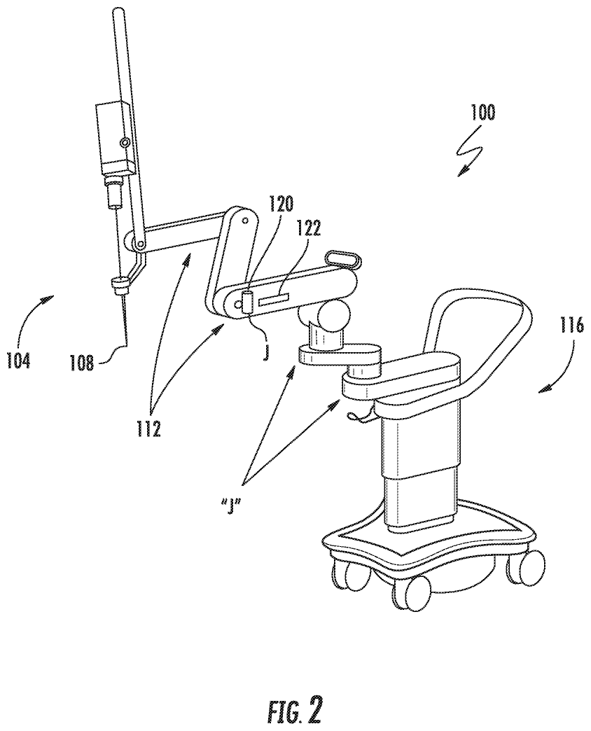

Referring to FIG. 2, the surgical robot 100 includes the robotic cart or tower 116 supporting the linkages 112 which support a tool 108. The linkages 112 includes one or more motor mechanisms 122 that are each associated with a respective joint “J” of the linkage 112 to manipulate the linkage 112 and/or the tool 108.

In use, the controller 200 (FIG. 1) transmits control signals to the surgical robot 100 to cause a motor mechanism 122 to apply a force about or to a respective joint “J”. Specifically, in response to a control signal, the surgical robot 100 delivers a power current to the motor mechanism 122. In response to the power current, the motor mechanism 122 applies a force to the joint “J”. As shown, the motor mechanism 122 applies a rotary force or torque to the joint “J”; however, the motor mechanisms 122 may apply other forces such as linear and/or compressive forces to joint “J”. Additionally or alternatively, the motor mechanisms 122 may be associated with any joint “J” of the linkages 112 of the surgical robot 100 to actuate the linkages 112 and/or tool 108 during a surgical procedure. A sensor 120 is coupled to the joint “J” and, in response to receiving force applied by the motor mechanism 122 to the joint “J”, the sensor 120 measures a torque about the joint “J” and transmits the measured torque measurement to the controller 200.

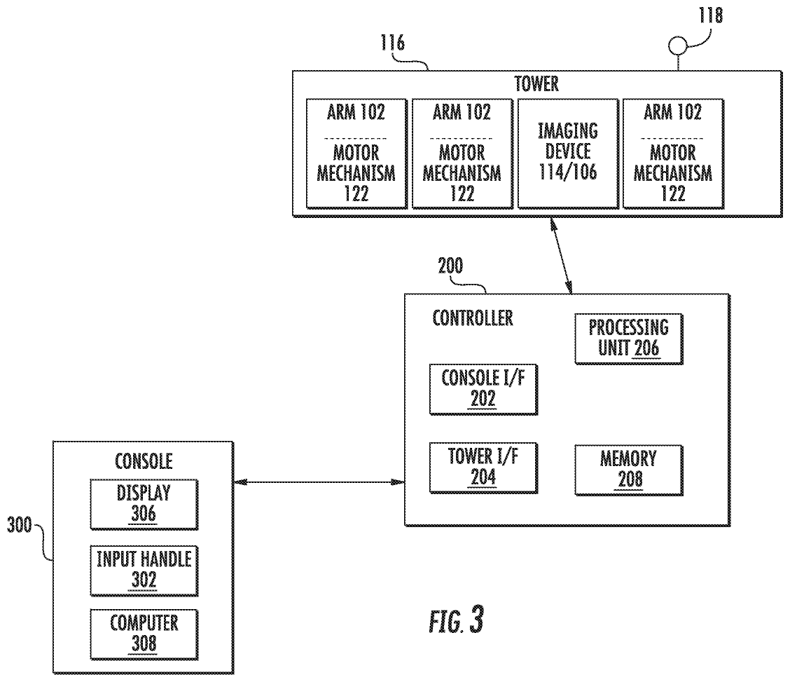

With reference to FIG. 3, communication between the surgical robot 100, the controller 200, and the console 300 are described in accordance with the present disclosure. The controller 200 is in communication with the tower 116 of the surgical robot 100 to provide instructions for operation, in response to input received from the console 300.

The controller 200 generally includes a processing unit 206, memory 208, a tower interface 204, and a console interface 202. The processing unit 206 includes a computer program stored in the memory 208 which functions to cause components of the tower 116, e.g., linkages 112, to execute desired movements according to movement defined by input handle 302 of the console 300. In this regard, the processing unit 206 includes any suitable logic control circuit adapted to perform calculations and/or operate according to a set of instructions. The processing unit 206 may include one or more processing devices, such as a microprocessor or other physical device capable of executing instructions stored in the memory 208 and/or processing data. The memory 208 may include transitory type memory, e.g., RAM, and/or non-transitory type memory, e.g., flash media or disk media. The tower interface 204 and consoles interface 202 communicate with the tower 116 and console 300, respectively, via either wireless configurations, e.g., Wi-Fi, Bluetooth, LTE, and/or wired configurations. Although depicted as a separate module, the console interface 202 and tower interface 204 may be a single component in other embodiments.

Continuing to refer to FIG. 3, the tower 116 includes a communications interface 118 that receives communications and/or data from the tower interface 204 of the controller 200 for manipulating motor mechanism 122 to thereby move the robotic arms 102 associated with the tower 116. The motor mechanism 122 may be located in one or more of robotic arms 102 and/or in the linkages 112. In embodiments, the motor mechanism 122 receives applications of power current for mechanical manipulation of the robotic arms 102, linkages 112, and/or tools 108 (FIG. 1). Mechanical manipulation of the robotic arms 102, linkages 112, and/or tools 108 may include the application of force from the motor mechanisms 122 to move a selected one of the robotic arms 102 and/or tools 108 coupled to a robotic arm 102, in response to instructions from the processing unit 206. For example, the motor mechanism 122 may be coupled to cables (not shown) to manipulate the robotic arms 102. Additionally, the motor mechanisms 122 may manipulate a variety of mechanisms to move the robotic arms 102 and/or tools 108. The tower 116 also includes an imaging device 106, 114, which captures real-time images and transmits data representing the images to the controller 200 via the communications interface 118.

To affect movement of the surgical robot 100, and in particular the devices of the tower 116, the console 300 further includes a computer 308. Each input handle 302 is coupled to the corresponding computer 308 and is used by the clinician to provide an input. In response to receiving clinician input from the input handle 302, the controller 200 transmits control signals to the tower 116, and the devices of the tower, to effect motion. The input handle 302 may be a handle, pedal, or a computer accessory, e.g., a keyboard, joystick, mouse, button, touch screen, switch, trackball. The display 306 displays images or other data received from the controller 200 to communicate data to the clinician. The computer 308 includes a processing unit and memory, which includes data, instructions and/or information related to the various components, algorithms, and/or operations of the tower 116 and can operate using any suitable electronic service, database, platform, cloud, or the like. The computer 308 may include processing units 206 which includes any suitable logic control circuit adapted to perform calculations and/or operate according to a set of instructions located in memory (not shown), as described similarly with reference to the controller 200.

For a detailed description of a surgical robot 100, reference may be made to U.S. Provisional Patent Application Ser. No. 62/345,032, filed Jun. 3, 2016 and entitled “Multi-Input Robotic Surgical System Control Scheme,” the entire disclosure of which is hereby incorporated by reference herein.

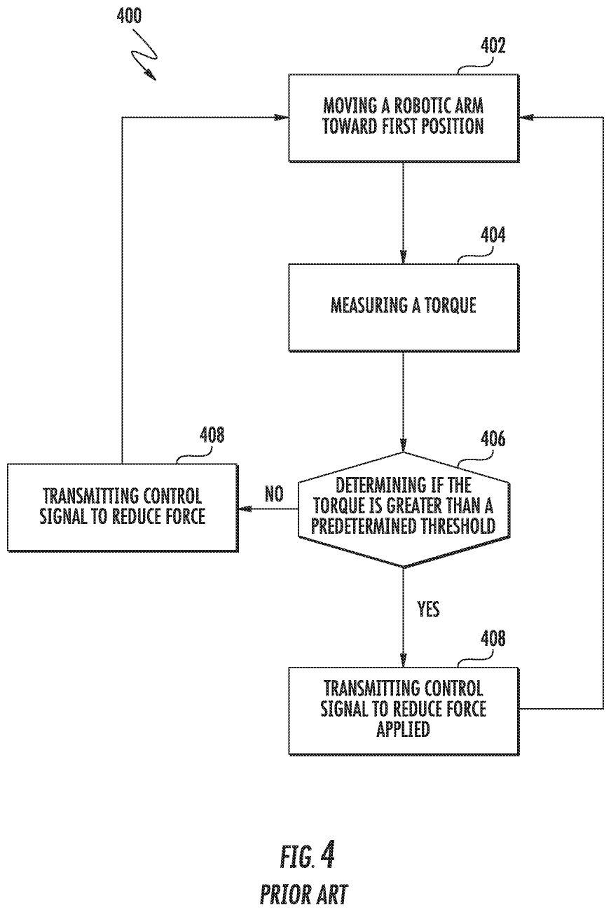

Referring to FIG. 4, a flow diagram of a prior art torque control process 400 (hereinafter “prior art process 400”) for limiting collision torque applied by the motor mechanisms 122 to a joint “J”, is described with reference to the surgical robot 100 of FIGS. 1 and 2. The prior art process 400 includes a clinician exerting a force on the input handle 302 sufficient to move the input handle 302 from a first position to a second position. In response to the motion of the input handle 302, the controller 200 scales the motion of the input handle 302 and transmits control signals to the tower 116 to move the robotic arm 102 from a first scaled position toward a second scaled position corresponding to the scaled motion of the input handle 302 (Step 402).

As the robotic arm 102 moves towards the second scaled position, the sensor 120 measures torque about the joint “J” and transmits torque measurements to the tower 116 (Step 404). The tower 116 receives a first torque from the sensor 120 at joint “J” and determines whether the first torque measurement is greater than a predetermined threshold (Step 406). If the first torque is greater than the predetermined threshold, the tower 116 reduces the force applied by the motor mechanism 122 to the joint “J” by a predetermined factor (Step 408). The predetermined factor may be any percentage value that the first torque is reduced when the first torque is greater than a predetermined threshold. Alternatively, if the first torque is less than the predetermined threshold, the controller 200 causes the tower 116 to maintain the force applied by the motor mechanism 122 to joint “J”, thereby continuing to move the robotic arm 102 toward the second scaled position (Step 410). The prior art process 400 is iteratively repeated until the robotic arm 102 reaches the second scaled position (Step 402).

With continued reference to FIG. 4, the prior art process 400 is executed via a local control circuit located in the tower 116. Alternatively, the prior art process 400 is stored as instructions in the memory 208 of the controller 200 and executed on the processing unit 206. As such, the controller 200 may iterate the prior art process 400 in response to receiving subsequent torque measurements. Where the subsequent torque measurements are greater than a predetermined threshold, the controller 200 may reduce the force applied by the motor mechanism 122 to the joint “J” (Step 408). Alternatively, where subsequent torque measurements are below a predetermined threshold, the controller 200 may continue transmitting control signals to the robotic arm to maintain force (Step 410).

Referring generally to prior art process 400, the reduction of force (S408) in response to sensing a torque with the sensor 120 (S404) can be used to backdrive a low-friction surgical robot 100. More particularly, when the sensed torque exceeds the predetermined threshold, the controller 200 transmits control signals to reduce the force applied by the surgical robot 100. However, when a surgical robot has to overcome non-negligible frictions during operation thereof, prior art process 400 does not contemplate compensating for such non-negligible frictions which are realized by the controller 200 as increased torque measurements. Additionally, prior art process 400 does not contemplate compensating for non-negligible inertial forces such as the initial force necessary to advance the surgical robot in a particular direction. These problems are addressed by the principles of the present disclosure, described herein.

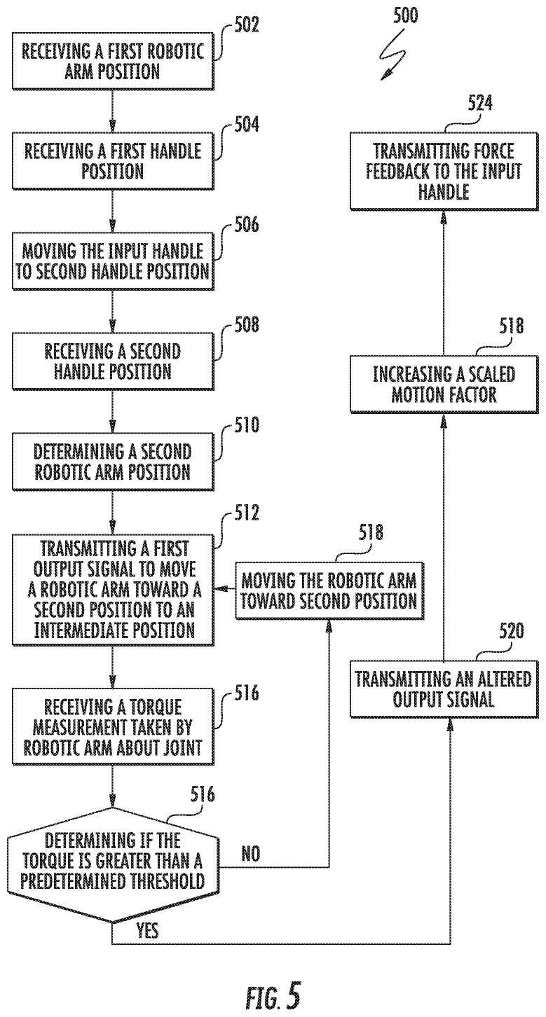

With reference to FIG. 5, a method 500 of collision handling for a robotic surgical system in a controller of the robotic surgical system (hereinafter “process 500”) for adjusting a second position (or end position) of the surgical robot 100 is disclosed in accordance with the present disclosure with reference to the robotic surgical system of FIGS. 1 and 2. Initially, the tower 116 transmits a first input signal including a first robotic arm position (or initial position) representative of the position and orientation of the robotic arm 102 relative to the tower 116 to the controller 200 (Step 502). The controller 200 also receives a first handle input including a first handle position from the computer 308 which includes information representative of the orientation and position of the input handle 302 within a workspace “W” of the console 300 (Step 504).

As the clinician moves the input handle 302 from the first handle position to a second handle position (Step 506) the clinician may apply longitudinal and/or rotational force to the input handle 302 to reposition the input handle 302 within the workspace “W” of the console 300. Once the input handle 302 is moved to the second handle position, a second handle input is transmitted to the controller 200. The controller 200 receives the second handle input from the computer 308 associated with the console 300, the second handle input including information representative of the orientation and position of the input handle 302 within the workspace “W” of the console 300 (Step 508). In response to receiving the second handle input, the controller 200 determines a second robotic arm position (or desired position) (Step 510).

To determine the second robotic arm position, the controller 200 measures a positional change (or path) between the first handle position and the second handle position. The path is defined as the direction and distance of the motion of the input handle 302 from the first input handle position to the second input handle position. The controller 200 then applies a scaling factor (S_{F}) to the path, and determines a second robotic arm position based on the scaled path (Step 510).

After determining the second robotic arm position the controller 200 sends control signals to the tower 116 which include a first output signal including commands to move the robotic arm 102 toward the second robotic arm position relative to the tower 116. The first output signal is received by the tower 116, and cause the tower 116 to transmit a power current to motor mechanism 122. As a result of receiving the power current, the motor mechanism 122 applies a force to the joint “J” to move the robotic arm 102 from the first robotic arm position toward the second robotic arm position (Step 512). As the robotic arm moves toward the second robotic arm position, the robotic arm 102 may collide with an obstruction, e.g., a surgical table, a wall defining the opening of the surgical cavity “S”, another robotic arm, and/or other objects located between the first robotic arm position and the second robotic arm position. To continue to move the robotic arm 102 towards the second robotic arm position, the motor mechanism 122 increases a force applied to the joint “J” to overcome the counter-force obstructing the robotic arm 102. Specifically, the tower 116 may increase the power current transmitted to the motor mechanism 122 to increase the force applied to the joint “J” by the motor mechanism 122. This increase in force may move, press on, and/or compress the obstruction with robotic arm 102.

As the robotic arm 102 is moved toward the second robotic arm position, the controller 200 receives torque measurements from the sensor 120 indicative of torque about the joint “J”, and the controller 200 compares the torque measurements to a predetermined threshold (Step 516). If a respective torque measurement is less than a predetermined threshold, the controller 200 continues to send control signals to move the robotic arm 102 towards the second position (Step 518). Additionally, as the robotic arm 102 moves toward the second robotic arm position, the controller 200 may subtract or otherwise compensate known resistances associated with moving the robotic arm 102 from the sensed torque measurements, while no collision or obstruction counteracts such motion. Specifically, as the robotic arm 102 is advanced toward the first position, the force determined by the controller 200 to be applied by the motor mechanisms 122 to move the robotic arm 102 may be increased or decreased to overcome known inertial or operational forces such as, without limitation, inertial forces, predetermined frictional forces associated with the components of the robotic system 100, gravitational forces which must be overcome to maintain the position or pose of the robotic arm 102 relative to the patient “P”, and the like. The force may be increased via multiple compensation techniques. For example, as torque measurements are received by the controller 200 from the sensor 120, the known forces associated with moving the robotic arm 102 in an unobstructed area may be subtracted from the sensed measurements. The resulting force measurements may subsequently be analyzed by the controller 200 prior to the controller 200 determining whether the torque measurements exceed the predetermined threshold.

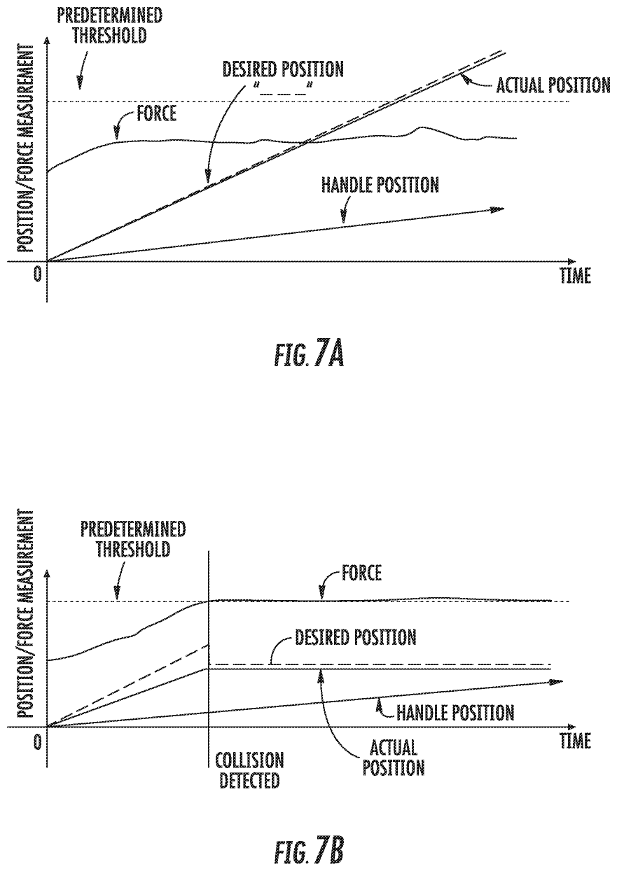

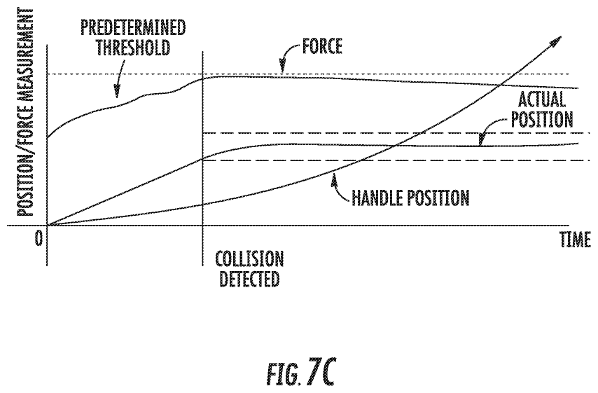

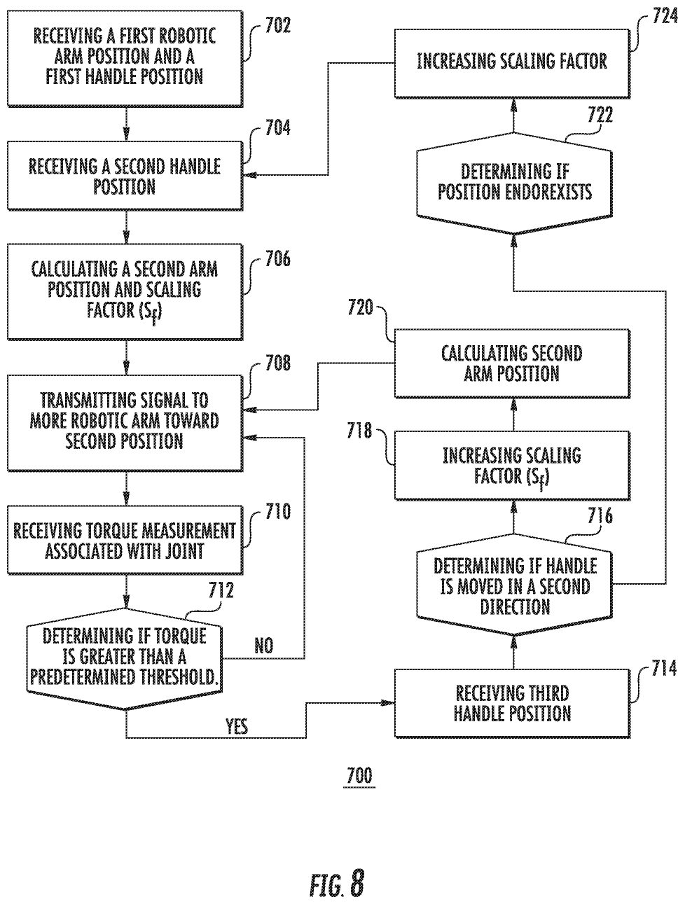

If the controller 200 determines that a respective torque measurement is greater than the predetermined threshold, the controller 200 alters the second robotic arm position in response to the respective torque measurement (Step 520), and generates an altered output signal. The altered output signal includes commands to move the robotic arm 102 to the altered second robotic arm position (or altered desired position). The altered second robotic arm position may be representative of the position and/or orientation of the robotic arm 102 relative to the tower 116 at the time the collision is detected (or a current position). It will be appreciated that the predetermined threshold may be indicative of a collision with an obstruction. Specifically, when the torque measurement exceeds the predetermined threshold, the controller 200 sets the second robotic arm position of the robotic arm 102 as an altered second robotic arm position, defined as a current position and orientation of the robotic arm 102. After setting the altered second robotic arm position as the current position and orientation of the robotic arm 102, the controller 200 sends the altered output signal to the tower 116, thereby causing the tower 116 to stop transmitting power to the motor mechanism 122 (Step 520). As shown in FIG. 7B, the process 500 effectively limits subsequent inputs from causing the robotic arm 102 to further compress or press on the obstruction significantly.

In addition to transmitting the altered output signal in response to determining that the torque measurement exceeds the predetermined threshold, the controller 200 increases the scaling factor applied to the motion of the input handle 302 (or scales down the motion) (Step 522). This increase of the scaling factor may, as perceived by the clinician engaging the surgical system 100, “clutch out” or otherwise reduce movement of the robotic arm 102 so as to appear to have significantly reduced or stopped the advance of the robotic arm 102 in the direction in which the arm is moving. Additionally, the placement of the sensor 120 along one or more joints “J” of the robotic arm 102 enables the controller 200 to receive sensor signals indicative of the motion of the components of the robotic arm 102 colliding with an object. These sensor signals, however, are not affected by and do not reflect a measurement of the forces associated with frictional forces associated with the drive train, e.g., the motor mechanism 122 and components translating forces transmitted by the motor mechanism 122 to the joints “J” of the robotic arm 102. Advantages of measuring force exerted by the portions of the surgical system 100 about joints “J” located distal relative to the motor mechanism 122, and more generally the drive components of the robotic arm 102, are discussed in commonly-owned U.S. Provisional Patent Application Ser. No. 62,554,208, filed Sep. 5, 2017, the contents of which are hereby incorporated in their entirety.

After increasing the scaling factor (Step 522) the controller 200 sends control signals to the input handle 302 to output force feedback against additional movement in the direction towards the second handle position (Step 524). Force feedback may be in the form of haptic feedback or other such tactile and/or sensory feedback to indicate to a clinician that the predetermined threshold has been exceeded. After the force feedback is transmitted to the user process 500 reiterates (Step 502) in response to continued movement of the input handle 302 by the clinician. Reiteration of process 500 may occur as the clinician continues to advance the input handle 302 to cause the robotic arm 102 to move in the first direction. When this occurs, the controller 200 recognizes that the scaling factor associated with translating the robotic arm 102 has increased, having determined that a collision has already occurred by advancing the robotic arm 102 in the first direction. The controller 200 may subsequently increase the scaling factor further causing the robotic arm 102 to appear to have “clutched out” while translating toward the first direction. The controller 200 is further configured to recognize that motion of the input handle 302 in a second direction different from the first direction does not require modification of the scaling factor used to determine a subsequently desired position of the robotic arm 102, and as such may reset or reduce the scaling factor. As a result, when a collision is recognized, the robotic arm 102 is translated or caused to translate away from the collision, the robotic arm 102, in response to clinician engagement of the input handles 302, advances in the second direction at a faster rate than when advanced during the collision. In embodiments, the scaling factor is reset to the value of the initial scaling factor (S_{f}) upon recognition by the controller 200 that the input handle 302 in being translated in the second direction, or away from the collision. As a result, the rate of translation of the robotic arm 102 away from the collision is increased immediately to allow the robotic arm 102 to be backdriven immediately.

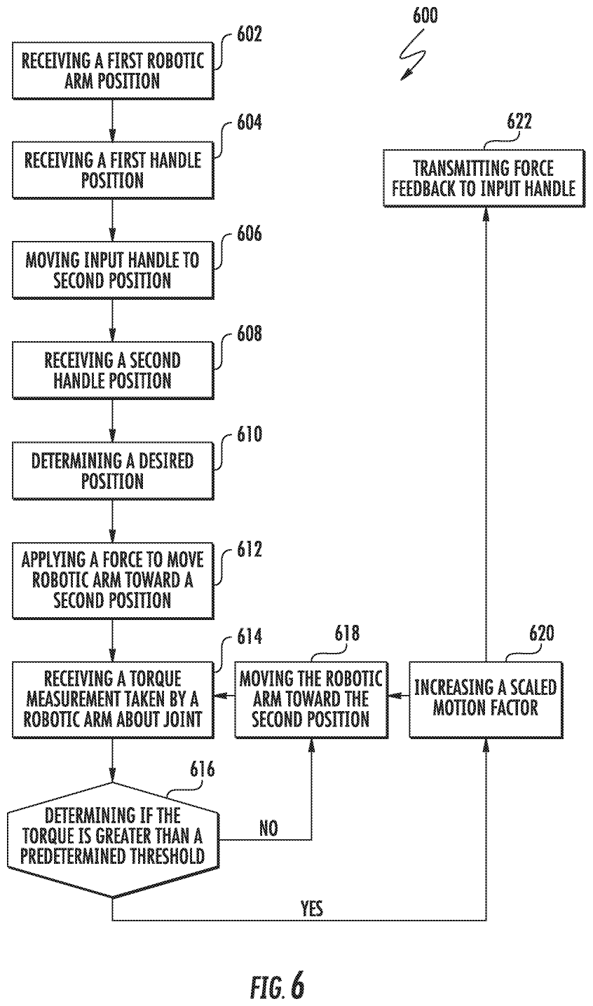

Referring to FIG. 6, another method of collision handling, for a robotic surgical system, in a controller of the robotic surgical system (hereinafter “process 600”) that adjusts desired positions of the robotic arm relative to the input handle 302 motion in the workspace “W”, in response to a torque exceeding a predetermined threshold, is shown and described. Initially, the controller 200 receives a first robotic arm position (or initial position) from the tower 116. Specifically, the tower 116 transmits a first input signal to the controller 200 which includes the first robotic arm position representative of the orientation of the robotic arm 102 relative to the tower 116 (Step 602). In addition, the controller 200 receives a first handle input including a first handle position from the computer 308. The first handle position includes information representative of the orientation and position of the input handle 302 within a workspace “W” of the console 300. Specifically, the computer 308 determines the position of the input handle 302 within the workspace “W” of the console 300 and transmits the first handle input to the controller 200 (Step 604).

A clinician may then move the input handle 302 from the first handle position to a second handle position relative to the workspace “W” (Step 606). The second handle position includes information representative of the orientation and position of the input handle 302 within a workspace “W” of the console 300. (Step 606). After the computer 308 determines the position of the input handle 302 within the workspace “W” of the console 300, the computer 308 transmits a second handle input, including the second handle position, to the controller 200 (Step 608).

In response to receiving the second handle input, the controller 200 determines a second robotic arm position (or desired position). Upon reception of the first and second handle inputs, the controller 200 measures a positional change (or path) between the first handle position and the second handle position. The path is defined as the direction and distance of the motion of the input handle 302 from the first handle position to the second handle position relative to the workstation “W”. The controller 200 then applies a scaling factor (S_{F}) to the path to determine a second robotic arm position (Step 610).

After determining the second robotic arm position (Step 610), the controller 200 sends a first output signal, including a second robotic arm position, to the tower 116 to cause the motor mechanism 122 to apply a force to the joint “J” (Step 612). In response to receiving the first output signal, the tower 116 causes the motor mechanisms 122 to move the robotic arm 102 toward the second robotic arm position (Step 612). During motion, the robotic arm 102 may collide with obstructions, e.g., a surgical table or walls defining a surgical cavity “S”. When the robotic arm 102 does not reach the second robotic arm position, the tower 116 may increase the power current transmitted to the motor mechanism 122, thereby causing the motor mechanism 122 to increase the force applied to the joint “J”. The increase in force applied to the joint “J” may cause the robotic arm 102 to compress, press on, or move the obstruction as the robotic arm 102 moves toward the second robotic arm position.

As the robotic arm 102 moves toward the second robotic arm position, the sensor 120 measures torque about joint “J” and transmits torque measurements to the controller 200. The controller 200 receives the torque measurements from the sensor 120 (Step 614) and compares the torque measurements to a predetermined threshold (Step 616). The predetermined threshold may be any torque value which is greater than desired or practicable for the surgical procedure being performed and/or may be indicative of a collision of the robotic arm 102 with an obstruction. If the torque measurement is less than the predetermined threshold, the controller 200 may send control signals to the robotic arm 102 to maintain or increase force applied by the motor mechanism 122 to the joint “J” (Step 618).

If a respective torque measurement is greater than the predetermined threshold, the controller 200 increases the scaling factor (S_{F}) applied to the motion (or path) of the input handle 302 for determining the second position (Step 620). Increasing the scaling factor (S_{F}) applied to the input handle 302 motion by the controller 200 simulates “slipping” or the reduced efficacy of the input handle 302 to move the surgical robot 100 toward the second position by requiring the input handle 302 to travel a greater distance than previously required to cause the robotic arm 102 to reach the second position. As process 600 is repeated, the scaling factor (S_{F}) is continually increased, thereby causing the controller 200 to transmit control signals which effectively limit the motion of the robotic arm 102 to the position of the robotic arm 102 when the predetermined threshold was reached, or shortly thereafter. As shown in FIG. 7C, this iterative reduction in force creates a positional limit in which the robotic arm 102 does not move beyond.

After increasing the scaling factor (Step 620) the controller 200 sends control signals to the computer 308 associated with the console 300 to send force feedback to the input handle 302 (Step 622). The force feedback transmitted to the input handle 302 may be in the form of vibration or other such tactile and/or sensory information. Transmission of the force feedback to the clinician gripping the input handle 302 indicates to the clinician that the force applied by the robotic arm 102 to continue to move in a particular direction is greater than the predetermined threshold, and that a collision has occurred with an obstruction in the surgical field.

Reiteration of process 500, similar to reiteration of process 400, may occur as the clinician continues to advance the input handle 302 to cause the robotic arm 102 to move in the first direction. When this occurs, the controller 200 recognizes that the scaling factor associated with translating the robotic arm 102 has increased, having determined that a collision has already occurred by advancing the robotic arm 102 in the first direction. The controller 200 may increase the scaling factor (S_{F}) causing the robotic arm 102 to appear to have “clutched out” while translating toward the first direction. The controller 200 is further configured to recognize that motion of the input handle 302 in a second direction different from the first direction does not require modification of the scaling factor used to determine a subsequently desired position of the robotic arm 102, and as such may reset the scaling factor (S_{F}) to a lesser scaling factor (S_{F}). As a result, when the clinician recognizes that a collision has occurred and attempts to change or otherwise reverse translation of the robotic arm 102, the robotic arm 102, in response to clinician engagement of the input handles 302, advances in the second direction at a faster rate than when advanced during the collision. Similar to process 400, in embodiments, when the scaling factor (Sr) is reset to the initial scaling factor (Sr) value after the controller 200 recognizes motion of the input handle 302 in the second direction, or away from the collision, the robotic arm 102 moves at the same rate as before the collision was detected.

Referring to FIG. 7A-7C, motion between a first position (or initial position) and a second position (or desired position) of a robotic arm 102 is shown in accordance with processes 500 and 600. In FIG. 7A, the robotic arm 102 is moved from the initial position (located at the origin “0” of the graph) toward the second position over a period of time. As the robotic arm 102 moves, the torque measurements received by the controller 200 do not exceed the predetermined threshold (Step 516, Step 616), and the robotic arm 102 continues to move toward the second position (Step 620).

With reference to FIG. 7B, the robotic arm 102 is moved toward a second position and a torque measurement exceeds the predetermined threshold as indicated. Specifically, as the robotic arm 102 is moved toward the second position, a torque measurement exceeds the predetermined threshold at the line labeled “collision detected”. Once the torque exceeds the predetermined threshold, the controller 200 transmits an altered output signal (Step 520) which causes the tower 116 to move the robotic arm 102 to the altered second position (or current position) (Step 520). As process 500 is iterated, the robotic arm 102 does not move beyond the altered second position in response to the input handle 302 motion in the corresponding direction of the second position.

With particular reference to FIG. 7C, the robotic arm 102 is moved toward a second position and a torque measurement exceeds the predetermined threshold. Specifically, as the robotic arm 102 is moved toward the second position, a torque measurement exceeds the predetermined threshold at the line labeled “collision detected”. Once the torque exceeds the predetermined threshold, the controller 200 increases the scaling factor (S_{F}) applied to the path (Step 620). As the input handle 302 continues to move toward the second position with torque measurements at or exceeding the predetermined threshold, the scaling factor is further increased, until the second position of the robotic arm 102 is effectively set as the actual position of the robotic arm 102.

Referring to FIG. 8, a process 700 of tracking movement of the input handle during a collision is described with reference to the surgical robot 100 of FIGS. 1 and 2. The process 700 includes the controller 200 receiving input indicative of a force exerted on the input handle 302 sufficient to move the input handle 302 in a first direction from a first position to a second position. The controller 200 additionally receives sensor signals from the workstation “W” including position information indicating the position of the input handle 302 when moved from the first position in the workspace “W” to the second position. Based on the sensor signals indicating the first position and the second position of the input handle 302 relative to the workspace “W”, the controller 200 determines the first direction (Step 704). The controller 200 scales the movement of the input handle 302, based on a scaling factor (S_{f}) (Step 706) and transmits control signals to the tower 116 to move the robotic arm 102 in the first direction from a first scaled position toward a second scaled position. The translation of the robotic arm 102 corresponds to scaled motion of the input handle 302 (Step 708).

As the robotic arm 102 moves toward the second scaled position, the sensor 120 measures torque about the joint “J” and transmits torque measurements to the tower 116 (Step 710). The tower 116 receives the torque measurements and determines whether the torque measurements are greater than a predetermined threshold (Step 712). If the measurements are less than the predetermined threshold, motion of the robotic arm 102 continues at the same rate with the value of the scaling factor (S_{f}) remaining constant.

If the torque measurements are greater than the predetermined threshold, indicating that the arm 102 is collided with an object in the surgical field “SF” (FIG. 1), the tower 116 receives a third handle position in response to continued movement of the input handle 302, toward a third position (Step 714). The tower 116 determines a second direction based on the motion of the input handle 302 from the second position to the third position. The tower 116 then compares the second direction to the first direction to determine the first direction is the same as the second direction (Step 716). In embodiments, the second direction may be opposite or substantially opposite the first direction, however in some embodiments, the second direction may be any direction away from the object.

When the tower 116 determines that the input handle 302 is continuing to move in the first direction, after determining the torque value is greater than the predetermined threshold, the tower 116 increases the scaling factor (S_{f}) (Step 718), causing the tower 116 to calculate a different second arm position (Step 720). By virtue of increasing the scaling factor (S_{f}), subsequent motion of the robotic arm 102 toward the second position requires translation of the input handle 302 beyond what would ordinarily be required (e.g., the input handle 302 must move twenty-percent further than previously required to achieve the same motion of the robotic arm 102). This additional translation causes the position of the input handle 302 in the workspace “W” to be offset relative to the scaled position of the robotic arm 102, the offset referred to herein as a position error. Additionally, by increasing the distance the input handle 302 must travel to achieve the same motion, the clinician may perceive that the robotic arm 102 is underperforming, indicating that a collision has occurred. The controller 200 may also transmit control signals to cause the input handle 302 to transmit force feedback, such as vibration, when the input handle 302 moves in the first direction once a collision is detected. The force feedback may increase in intensity as the input handle 302 is further advanced in the first direction once the collision is detected, and similarly, may decrease as the input handle 302 is moved in a second direction by the clinician.

In response to the tower 116 determining the input handle 302 is moving in the second direction, away from the collision, the tower 116 determines if a position error exists between the position of the input handle 302 within the workspace “W” and the robotic arm 102 in the surgical site “S” (FIG. 1). If no position error is determined to exist (Step 722), process 700 repeats Step 706 with the tower 116 calculating a second arm position and a scaling factor (S_{f}). More particularly, the tower 116 sets the scaling factor (S_{f}) to the initial scaling factor (Step 724) and continues to Step 704 to reiterate process 700 when no position error exists and the robotic arm 102 is moving away from the object which it previously collided with.

When a position error is determined to exist (Step 722), the tower 116 increases the scaling factor (S_{f}) and calculates a second position based on the increased scaling factor. Process 700 reiterates and, as the input handle 302 continues to move in the second direction, the scaling factor (S_{f}) continues to increase until the scaling factor (S_{f}) is the same scaling factor (S_{f}) used to calculate positions when collisions between the robotic arm 102 and objects in the surgical field “SF” are not detected.

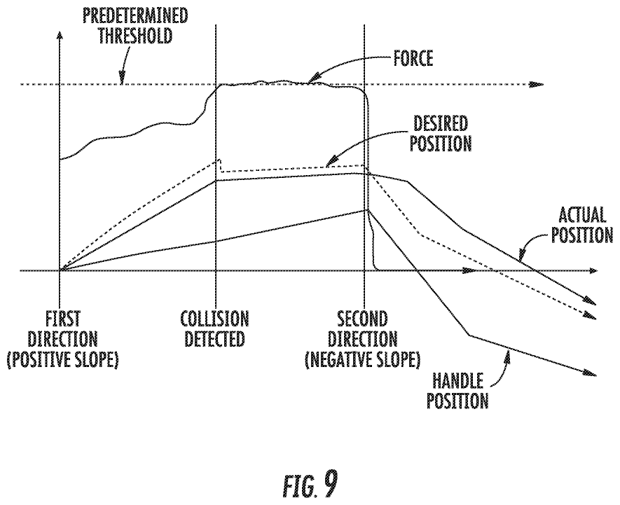

Referring to FIG. 9, the position diagram of the position of an input handle 302 and the position of the robotic arm 102 translated over time illustrates modification of the scaling factor (S_{f}) based position errors detected during a collision. Specifically, FIG. 9 illustrates operation of the robotic arm 102 during a collision, both when the input handle 302 is moved in a first direction, causing the input handle 302 to move into a collision with a foreign object as well as in a second direction, causing the robotic arm 102 to move away from the collision. Reference is made to calculation of the scaling factor (S_{f}) and motion of both the robotic arm 102 and the input handle 302 without regard to any particular scaling factor.

As the input handle 302 is translated through workstation “W” in a first direction, the scaling factor (S_{f}) is set to an initial value. Once the robotic arm 102 collides with an object in the surgical field “S”, an increase in measured torque is detected. In response to the increase in measured torque, the tower 116 increases the scaling factor (S_{f}) to a value greater than 1. The scaling factor (S_{f}) is continually increased until the input handle 302 is moved in a second direction away from the object which the robotic arm 102 collided with. As the scaling factor (S_{f}) is increased, a position error between the robotic arm 102 and the input handle 302 is calculated by the tower 116. As motion of the input handle 302 continues in the first direction, the tower 116 updates the calculated position error, which increases until the input handle 302 is moved in the second direction.

Upon detecting the input handle 302 is moved in the second direction, the tower 116 recalculates the position error to reduce the scaling factor (S_{f}). As the input handle 302 is moved in the second direction, away from the object the arm 102 collided with, the position error decreases until the position of the input handle 302 and the position of the robotic arm 102 are substantially aligned within an acceptable threshold. When the position error is eliminated or within the acceptable threshold, the scaling factor (S_{f}) is set to the initial value, restoring normal operational movement of the robotic arm 102. By gradually restoring the scaling factor (S_{f}) to the initial value, the input handle 302 is allowed to return to a default or center position within the workspace “W”. More specifically, as the robotic arm 102 moves in the second direction, the tower 116 decreases the scaling factor (S_{f}) used to calculate the scaled motion of the robotic arm 102. The decrease in the scaling factor (S_{f}), in turn, causes the tower 116 to transmit control signals to move the arm 102 at greater rates until the arm 102 moves at the initial rate of motion. For a detailed description of motion of the robotic arm 102, reference may be made to U.S. Patent Application Publication No. 2017/0224428, filed on Sep. 21, 2015, entitled “Dynamic Input Scaling for Controls of Robotic Surgical System,” the entire contents of which are herein incorporated by reference.

The technology of the present disclosure provides novel systems, methods, and arrangements to detect and alter control signals sent from the controller 200 after detecting a collision between elements of the surgical robot 100 and/or components external to the surgical robot 100. Though detailed descriptions of one or more embodiments of the disclosed technology have been provided for illustrative purposes, various alternatives, modifications, and equivalents will be apparent to those of ordinary skill in the art without varying or departing from the spirit of the invention. For example, while the embodiments described above refer to particular features, components, or combinations thereof, such features, components, and combinations may be substituted with functionally equivalent substitutes which may or may not contain the elements as originally described.

Further, while the disclosed embodiments contemplate location of a controller 200 external to a surgical robot 100, it is contemplated that the controller 200 may be located within the surgical robot 100, or alternatively that elements of the robotic surgical system 1 may include circuitry which executes the described force measurements and calculations independent of a controller 200.

As detailed above, the console 300 is in operable communication with the surgical robot 100 to perform a surgical procedure on a patient; however, it is envisioned that the console 300 may be in operable communication with a surgical simulator (not shown) to virtually actuate a surgical robot and/or tool in a simulated environment. For example, the robotic surgical system 1 may have a first mode in which the console 300 is coupled to actuate the surgical robot 100 and a second mode in which the display 306 is coupled to the surgical simulator to virtually actuate a robotic surgical system. The surgical simulator may be a standalone unit or be integrated into the controller 200. The surgical simulator virtually responds to a clinician interfacing with the console 300 by providing visual, audible, force, and/or haptic feedback to a clinician through the console 300. For example, as a clinician interfaces with the input handles 302, the surgical simulator moves representative tools that are virtually acting on tissue. It is envisioned that the surgical simulator may allow a clinician to practice a surgical procedure before performing the surgical procedure on a patient. In addition, the surgical simulator may be used to train a clinician on a surgical procedure. Further, the surgical simulator may simulate “complications” during a proposed surgical procedure to permit a clinician to plan a surgical procedure.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

It is contemplated that the systems and methods described in the present disclosure may be implemented in robotic surgical systems which implement telemanipulation techniques. “Telemanipulation” refers generally to the operation of a surgical system from a remote console by a clinician. By way of example, a telemanipulation may be a remote adjustment of the position of a robotic surgical instrument relative to a patient. Alternatively, telemanipulation may include an individual causing a robotic surgical instrument to perform one or more functions which the instrument is capable of doing.

What is claimed is:

1. A method of collision handling for a robotic surgical system in a controller of the robotic surgical system, the method comprising:
receiving a first handle input and a second handle input from a console of the robotic surgical system;
calculating a desired position of a robotic arm of a surgical robot in response to receiving the first and second handle inputs;
transmitting a first output signal to the surgical robot to move the robotic arm toward the desired position;
receiving a force measurement from the surgical robot as the robotic arm moves towards the desired position; and
recalculating the desired position of the robotic arm of the surgical robot when the force measurement is greater than a predetermined threshold.

2. The method according to claim 1, wherein the force measurement is taken at a joint of the robotic arm.

3. The method according to claim 1, wherein the force measurement is a torque measurement.

4. The method according to claim 1, further comprising transmitting a second output signal to continue moving the robotic arm toward the desired position when the force measurement is less than the predetermined threshold.

5. The method according to claim 4, further comprising receiving a subsequent force measurement and recalculating the desired position of the robotic arm in response to receiving the subsequent force measurement.

6. The method according to claim 5, wherein recalculating the desired position further includes setting the desired position as a current position of the surgical robot when the subsequent force measurement is greater than the predetermined threshold.

7. The method according to claim 1, further comprising transmitting control signals to transmit force feedback to an input handle when the force measurement is greater than the predetermined threshold.

8. The method according to claim 7, wherein transmitting control signals includes transmitting control signals to transmit at least one of haptic feedback, tactile feedback, or sensory feedback to the input handle when the force measurement is greater than a predetermined threshold.

9. A method of collision handling for a robotic surgical system in a controller of the robotic surgical system, the method comprising:
determining a desired position of a robotic arm;
transmitting an output signal to move the robotic arm towards the desired position;
receiving a force measurement from the robotic arm as the robotic arm moves towards the desired position; and
scaling down the output signal to move a location of the desired position when the force measurement is greater than a predetermined threshold.

10. The method according to claim 9, further comprising receiving a first input signal and a second input signal, and wherein determining the desired position of the robotic arm occurs in response to receiving the first and second input signals.

11. The method according to claim 8, further comprising transmitting additional output signals to move the robotic arm toward the desired position when the force measurement is less than the predetermined threshold.

12. The method according to claim 9, further comprising transmitting control signals to apply force feedback to an input handle when the force measurement is greater than the predetermined threshold.

13. A method of collision handling for a robotic surgical system with a controller of the robotic surgical system, the method comprising:
determining a desired position of a robotic arm;
transmitting a first output signal to move the robotic arm towards the desired position;
receiving a force measurement from the robotic arm as the robotic arm moves towards the desired position; and
transmitting an altered output signal when the force measurement is greater than a predetermined threshold.

14. The method according to claim 13, further comprising receiving a first handle input and a second handle input.

15. The method according to claim 14, wherein determining the desired position of the robotic arm further includes determining the desired position of the robotic arm in response to receiving the first and second handle input.

16. The method according to claim 13, wherein transmitting an altered output signal causes the robotic arm to move to an altered desired position in response to receiving the altered output signal.

17. The method according to claim 13, further comprising transmitting control signals to move the robotic arm towards the desired position when the force measurement is less than the predetermined threshold.

18. The method according to claim 13, further comprising transmitting control signals to apply force feedback to an input handle when the force measurement is greater than the predetermined threshold.
